(19) United States
(12) Patent Application Publication
(10) Pub. No.: US 2018/0296495 A1
(43) Pub. Date: Oct. 18, 2018

(54) A FAST ACTING ORALLY DISINTEGRATING FILM FOR ADMINISTRATION OF LOCAL ANESTHESIA

(71) Applicant: TAHO PHARMACEUTICALS LTD., TAIPEI (TW)

(72) Inventors: Catherine Lee, West Linn, OR (US); Chien-Chiao Wang, New Taipei City (TW); Fang-Chu Lin, Kaohsiung City (TW)

(73) Assignee: TAHO PHARMACEUTICALS LTD., TAIPEI (TW)

(21) Appl. No.: 15/761,811

(22) PCT Filed: Jun. 30, 2016
(86) PCT No.: PCT/US16/40228

§ 371 Date: Mar. 21, 2018

(51) Int. Cl. A61K 9/70; A61K 9/00; A61K 31/164; A61P 23/02

(52) U.S. Cl. CPC A61K 9/7007; A61P 23/02; A61K 31/164; A61K 9/0056

(57) ABSTRACT

A fast acting orally disintegrating film (ODF) for administration of local anesthetic for alleviating physical and psychological discomfort in the oral cavity during procedures such as dental procedures or for relieving pain generally such as toothaches. The ODF comprises an active pharmaceutical ingredient such as lidocaine free base or a pharmaceutically acceptable salt thereof in a therapeutically acceptable amount such as about 24 mg, at least one primary hydrophilic film forming polymer, at least one secondary hydrophilic film forming polymer, wherein the ratio of the primary hydrophilic film forming polymer to the secondary hydrophilic film forming polymer is about 1:1 to about 20:1 by weight. The ODF further comprises a plasticizer wherein the ratio of the total weight of primary and secondary hydrophilic film forming polymer to the weight of the plasticizer is about 4:1 to about 4:3.

### CROSS-REFERENCE TO RELATED APPLICATION(S)

The present application is a national stage patent application to PCT/US16/40228 with filing date of Jun. 30, 2016 entitled “A FAST ACTING ORALLY DISINTEGRATING FILM FOR ADMINISTRATION OF LOCAL ANESTHESIA.”

### FIELD OF THE INVENTION

The present invention relates to a fast acting orally disintegrating film (ODF) containing lidocaine, uses thereof and a method of producing the ODF.

### BACKGROUND OF THE INVENTION

Local anesthetics are drugs used for mitigating pain sensation at site of administration. An example of a local anesthetic is lidocaine with chemical name acetamide 2-(diethylamino)-N-(2,6-dimethylphenyl).

Lidocaine is often used in dentistry to alleviate physical discomfort as well as psychological tension in patients during dental procedures such as oral surgery, tooth extraction, root canal as well as to mitigate pain from toothaches, oral ulcers, cold sores or teething etc. . . . For the dental procedures, lidocaine is commonly administered by injection such as “Xylocaine® Cartridge for Dental Use” (Fujisawa Pharmaceutical Co., Ltd.). However, the invasive nature of the injection can itself cause both physical as well as psychological discomfort for the patient. In addition, for applications aside from dental procedures such as alleviation of pain from toothaches, a non-invasive form of administration is usually preferred. Furthermore, a method of administering lidocaine that is quick to provide therapeutic effect is beneficial for both doctors as well as patients. Therefore, there is a need for a method for administration of local anesthetic that is both non-invasive as well as fast acting.

### SUMMARY OF THE INVENTION

Accordingly, it is one objective of the present invention to provide a fast acting orally disintegrating film (ODF) for administration of local anesthetic.

The present invention provides a fast acting ODF comprising lidocaine free-base or a pharmaceutically acceptable salt thereof, at least one primary film-forming polymer, at least one secondary film-forming polymer and at least one plasticizer, wherein the primary film-forming polymer and the secondary film-forming polymer are present at a ratio of about 1:1 to 20:1 by weight and wherein the primary film-forming polymer and the secondary film-forming polymer are hydrophilic.

In some embodiments, the pharmaceutically acceptable lidocaine salt comprises lidocaine hydrochloride. In another embodiment, the lidocaine is present at an amount comprising at least about 10% to about 20% by dry weight of said film. In yet another embodiment, the primary film-forming polymer comprises hydroxylpropyl cellulose (HPC) or hydroxylpropyl methylcellulose (HPMC). In some embodiments, the secondary film-forming polymer comprises HPC, HPMC, pullulan and/or povidone (PVP). In another embodiment, the HPMC comprises HPMC 3 cps, HPMC 6 cps or HPMC 15 cps. In yet another embodiment, the PVP comprises PVP K-30 or PVP K-90. In some embodiments, the at least one plasticizer comprises polyethylene glycol (PEG), glycerol or Tween 20. In another embodiment, the PEG comprises PEG 400, PEG 4000 and/or PEG 6000. In some embodiments, the dry weight of the primary polymer as compared to dry weight of the secondary polymer is in a ratio of about 1:1 to about 7:1. In another embodiment, the dry weight of the primary and secondary polymers as compared to the dry weight of the plasticizer are in a ratio of about 4:1. In yet another embodiment, the primary polymer comprises HPMC 6 cps and the plasticizer comprises a plasticizer with high viscosity above about 500 cps but below about 2000 cps and low molecular weight of below about 1000 Daltons. In some embodiments, the primary polymer comprises HPMC 15 cps and the plasticizer comprises a plasticizer with low viscosity below about 200 cps but above 3 cps and high molecular weight of above about 5000 Daltons but below about 5,000,000 Daltons.

In other embodiments, the primary polymer comprises HPMC 15 cps, the secondary polymer comprises HPMC 3 cps or HPMC 6 cps and the plasticizer comprises glycerol or PEG 6000, wherein the primary film-forming polymer, the secondary film-forming polymer and the plasticizer are present at a ratio of about 2:2:1 to about 7:1:2 by weight. In yet another embodiment, the primary polymer comprises HPMC 15 cps, the secondary polymer comprises HPMC 3 cps, HPMC 6 cps or pullulan and the plasticizer comprises PEG 6000, wherein the primary film-forming polymer, the secondary film-forming polymer and the plasticizer present at a ratio of about 3:1:1 by weight. In some embodiments, the primary polymer comprises HPMC 6 cps, the secondary polymer comprises HPMC 3 cps or pullulan and the plasticizer comprises glycerol wherein the primary film-forming polymer, the secondary film-forming polymer and the plasticizer present at a ratio of about 3:1:1 by weight. In another embodiment, the primary polymer comprises HPMC 6 cps, the secondary polymer comprises PVP K-90 and the plasticizer comprises PEG 4000 wherein the primary film-forming polymer, the secondary film-forming polymer and the plasticizer present at a ratio of about 7:1:2 by weight.

In some embodiments, the fast acting ODF disintegrates within about 60 seconds of administration. In other embodiments, the dissolution rate of the fast acting ODF allows for release of about 90% of the lidocaine within about 5 minutes to about 10 minutes of administration. In yet another embodiments, the permeation rate of the fast acting ODF allows about 0.8 to about 1.7 mg/cm² of lidocaine to permeate through the targeted area treated with the ODF within about 5 minutes of administration. In some embodiments, no trace of organic solvent is present in the composition.

In some embodiments, the orally disintegrating film is produced by a method comprising the steps of mixing the local anesthetic, the two polymers and the at least one plasticizer in an aqueous solution, transferring the aqueous solution to a surface of a suitable carrier material and drying the aqueous solution on surface of the carrier material to form a film.

The present invention also provides a method for providing local anesthesia to a patient in need, the method comprising: orally administering to the patient in need thereof an effective amount of a composition comprising lidocaine free-base or a pharmaceutically acceptable salt thereof, at least one primary film-forming polymer, at least one secondary film-forming polymer and at least one plasticizer, wherein the primary film-forming polymer and the secondary film-forming polymer are present at a ratio of about 1:1 to 20:1 by weight and wherein the primary film-forming polymer and the secondary film-forming polymer are hydrophilic. In some embodiments, the method is used during oral surgery or dental treatment.

### BRIEF DESCRIPTION OF DRAWINGS

FIG. 1 illustrates the dissolution profiles of Lidocaine ODF of the present invention and Trachisan® Sore Throat Lozenges (Engelhard Arzenimittel GmbH & Co. KF, Germany) in 0.1 N HCL (pH 1.2).

FIG. 2 illustrates the dissolution profiles of Lidocaine ODF of the present invention and Trachisan® Sore Throat Lozenges (Engelhard Arzenimittel GmbH & Co. KF, Germany) in phosphate buffer (pH 4.5).

FIG. 3 illustrates the dissolution profiles of Lidocaine ODF of the present invention Trachisan® Sore Throat Lozenges (Engelhard Arzenimittel GmbH & Co. KF, Germany) in phosphate buffer (pH 6.8).

FIG. 4 illustrates the dissolution profiles of Lidocaine ODF of the present invention and Trachisan® Sore Throat Lozenges (Engelhard Arzenimittel GmbH & Co. KF, Germany) in water.

FIG. 5 illustrates the in vitro permeation profiles of Lidocaine ODF of the present invention and Xylocaine® Jelly 2% (Recipharm Karlskoga AB, Sweden) in water.

FIG. 6 illustrates a method for preparing the ODF of the present invention.

### DETAILED DESCRIPTION OF THE INVENTION

As used in this specification and in claims which follow, the singular forms “a”, “an” and “the” include plural referents unless the context clearly indicates otherwise. Thus, for example, reference to “an ingredient” includes mixtures of ingredients, reference to “an active pharmaceutical agent” includes more than one active pharmaceutical agent, and the like.

The terms “active agent,” “pharmacologically active agent” and “drug” are used interchangeably herein to refer to a chemical material or compound that includes a desired pharmacological, physiological effect and include agents that are therapeutically effective. The terms also encompass pharmacologically active derivatives and analogs of those active agents specifically mentioned herein, including, but not limited to, salts, esters, amides, sulfates, prodrugs, active metabolites, inclusion complexes and the like.

As used herein, the term “about” as a modifier to a quantity is intended to mean + or −10% inclusive of the quantity being modified.

As used herein, the term “disintegrate”, “disintegrating”, and “disintegrated” is intended to mean dispersing or otherwise breaking apart into small pieces that are undetectable by the naked eye.

As used herein, the term “dissolution” is intended to mean disintegration as defined above followed by further breaking down of the small pieces so as to free active pharmaceutical ingredient from the excipient or any other components of the present invention for mucosal absorption.

The term “effective amount” or “a therapeutically effective amount” of a drug or pharmacologically active agent is intended to mean a nontoxic but sufficient amount of the drug or active agent for providing the desired therapeutic effect. The amount that is “effective” will vary from subject to subject, depending on the age and general condition of the individual, the particular active agent or agents, and the like. An appropriate “effective” amount in any individual case may be determined by one of ordinary skill in the art using routine experimentation.

As used herein, the term “hydrophilic” refers to substances that have strongly polar groups that readily interact with water.

As used herein, the terms “local anesthetic” and “topical anesthetic” are interchangeable. A local anesthetic is a drug which provides local numbness or pain relief.

It will be understood that the term “film” comprises films and sheets, in any shape, including rectangular, square or other shapes most appropriate for a specific application. The films described herein may be of any desired thickness and size suitable for the intended use. For example, a film of the present invention may be sized and shaped so that it may be easily placed into the oral cavity of the user to target a specific administration site for effective, localized delivery of lidocaine. In addition, some films may have a relatively thin thickness of from about 10 to about 500 micrometers while others may have a somewhat thicker thickness of from about 500 to about 10000 micrometers. The variation of ODF thickness is desirable so that ODF of the present invention may be able to deliver a higher or lower lidocaine dosage for a particular treatment area. In addition, the term “film” includes single layer compositions as well as multi-layer compositions, such as laminated films, coatings on films and the like.

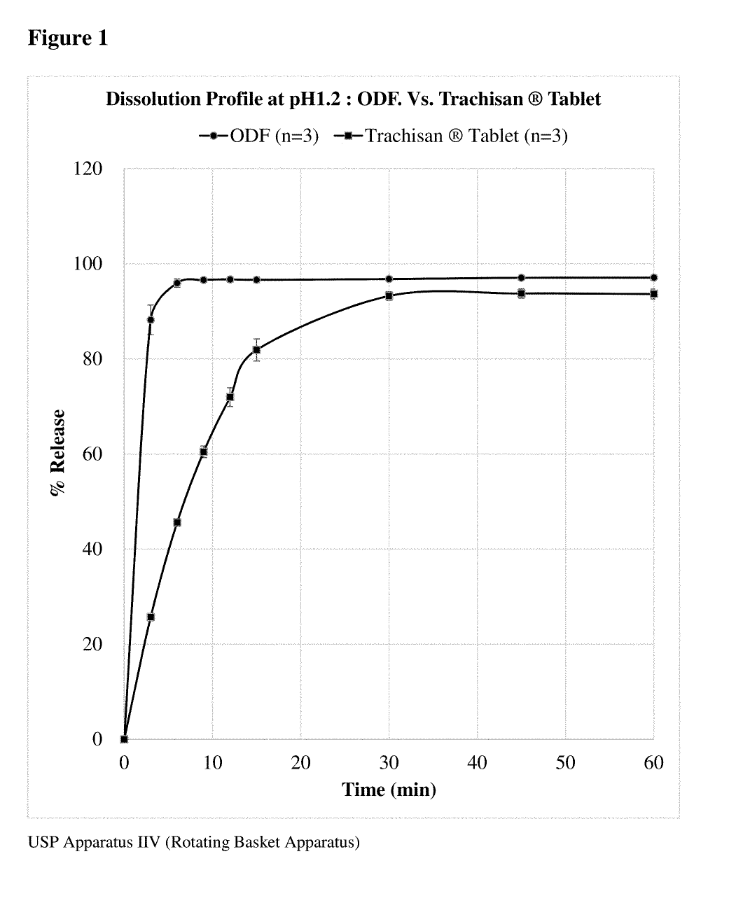

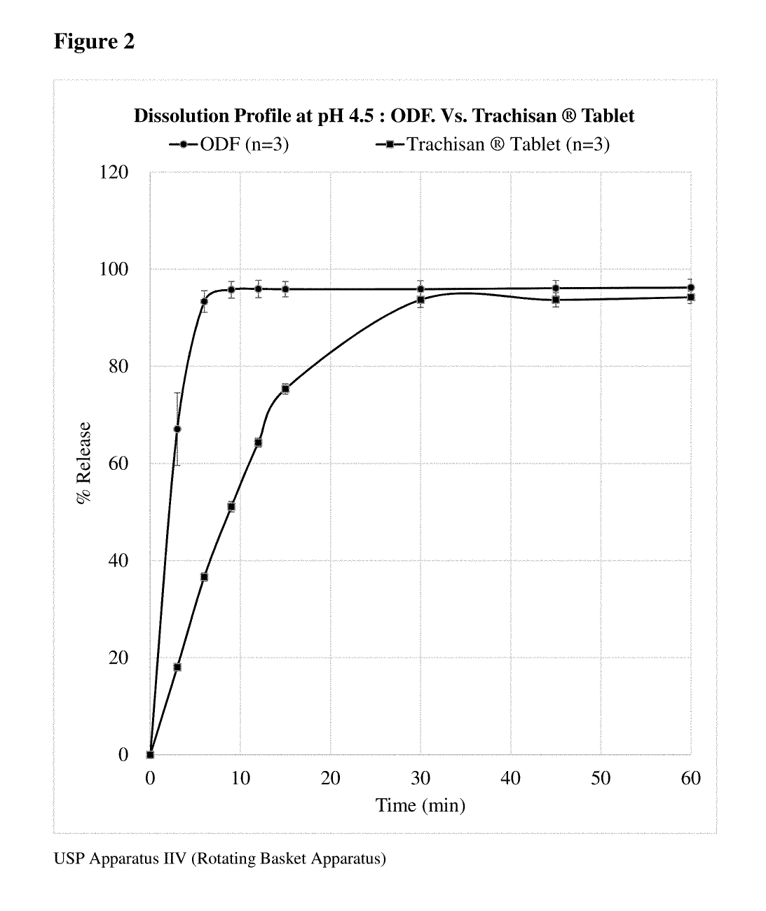

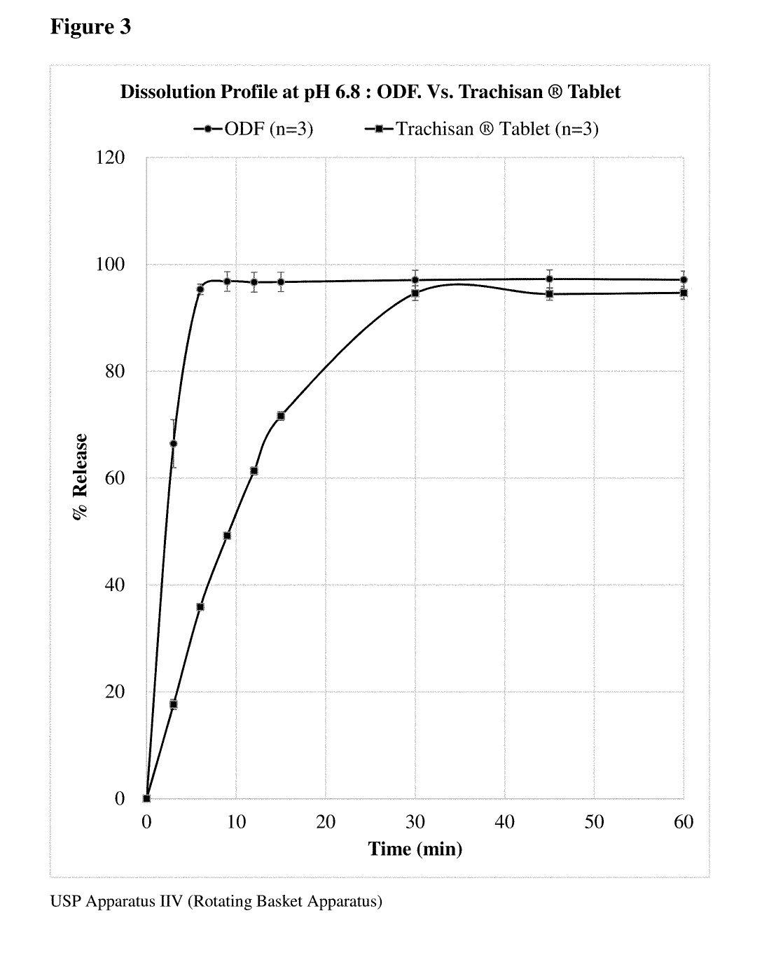

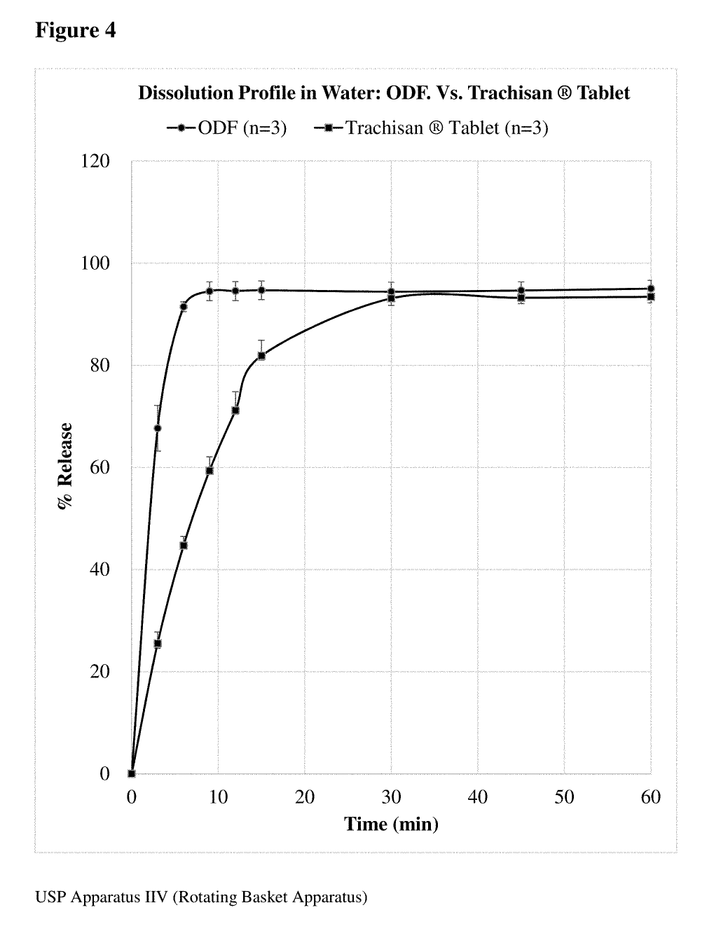

The present invention discloses a fast acting ODF comprising lidocaine as pharmaceutically active ingredient designed to allow administration in the absence of water or fluid intake. The ODF of the present invention is fast acting due to characteristics such as fast disintegration, dissolution and permeation rates. Specifically, the fast acting ODF of the present invention disintegrates in the saliva in less than about 60 seconds. It also provides higher dissolution and permeation rates than currently commercially available non-invasive means of administration such as gels and lozenges as will be discussed in connection with Examples 2 and 3 as well as FIGS. 1-5 below.

The fast acting characteristics of the ODF aids in rapid onset of desired anesthetic effects. As discussed in the background section, it is desirable for the ODF to be fast acting in order to render dental procedures as short and efficient as possible and to provide relief from pain quickly, benefiting both doctors and patients, especially those patients that have an aversion to dental procedures. The fast acting property becomes even more important when multiple administrations of lidocaine are required.

Another advantage of the ODF of the present invention is that the ODF form allows customization of its size and shape to better target the administration site and administration purpose. Furthermore, the ODF described herein is capable of delivering localized high dosage with little systemic absorption, minimizing adverse side effects associated with the systemic absorption of local anesthetics.

In an embodiment, lidocaine is in the form of lidocaine free-base. In other embodiments, the local anesthetic may be any pharmaceutically acceptable salts and prodrugs of lidocaine, for example, the hydrochloride, hydrobromide, acetate, citrate, or sulfate salt.

In some embodiments, the ODF described herein contains from about 10% to about 60% of the local anesthetic by total weight of the formulation. More specifically, the lidocaine ODF described herein contains about 15%, 20%, 25%, 30%, 35%, 40%, 45%, 50%, 55% or 60% of the lidocaine by total weight of the formulation.

The lidocaine ODF of the present invention further comprises one or more film-forming polymers. The film-forming polymers preferably possess one or more of the following attributes: readily available, inexpensive, hydrophilic, non-toxic, tasteless and hypoallergenic. In addition, the film-forming polymers preferably dissolve easily in solvents such as water and disintegrates and dissolves rapidly in the buccal cavity.

The film-forming polymers lend crucial properties to the ODF of the present invention such as film-forming, extensibility, homogeneity and smoothness of surface properties as will be discussed more specifically in connection with Example 1. The film-forming property is based on level of breakages in an ODF as result of film-forming process. The extensibility property is based on level of breakages in an ODF as result of pulling or bending of the ODF that usually occur during normal handling conditions. The homogeneity property is based on level of layer separation in the ODF formed during the film-forming process. The smoothness of surface property is based on level of precipitates or air bubbles in the ODF that form during the film-forming process.

Exemplary film-forming polymers possessing one of more of the preferred properties listed above include, but is not limited to, hydroxypropyl methylcellulose (HPMC), hydroxypropylcellulose (HPC), pullulan, povidone (PVP), hydroxyethyl cellulose, sodium carboxy methyl cellulose, starch and derivatives thereof. Examples of HPMC are HPMC with viscosity from about 3 cps to about 100,000 cps and, more specifically, HPMC 3 cps, HPMC 6 cps and HPMC 15 cps. Examples of PVP comprise PVP K-12 to PVP K-120, and, more specifically, PVP K-30 and PVP K-90.

The ODF of the present invention may comprise a primary film-forming polymer (primary polymer) as well as a secondary film-forming polymer (secondary polymer) wherein the primary polymer comprises equal or greater weight in comparison to the secondary polymer. Inclusion of both a primary as well as secondary polymer with differing properties such as viscosity and molecular weight at appropriate ratios plays an important role in ODFs with desirable properties such as film-forming property, homogeneity and smoothness of surface as will be discussed in connection with Example 1 and Tables 1A and 1B below.

The ratio of primary to secondary polymer may range from about 1:1 to about 20:1 by weight %. In certain embodiments, the primary polymer and the secondary polymer are present at a ratio of about 1:1 to about 10:1 by weight %. In some embodiments, the primary polymer and the secondary polymer are present at a ratio of about 1:1, 2:1, 3:1, 3:2, 4:1, 5:1, 5:2, 5:3, 6:1, 7:1, 8:1, 9:1 or 10:1 by weight %.

In certain embodiments, both the primary polymer and the secondary polymer are hydrophilic wherein the primary polymer may comprise HPC or HPMC while secondary polymer may comprise HPC, HPMC, PVP and/or pullulan. More specifically, in some embodiments, the primary polymer comprises HPMC 6 cps or HPMC 15 cps while the secondary polymer comprises HPMC 3 cps, HPMC 6 cps, PVK-30, PVK-90 or Pullulan. In other embodiments, the primary polymer comprises HPMC 15 cps while the secondary polymer comprises HPMC 3 cps, HPMC 6 cps, Povidone K-30, Povidone K-90 or Pullulan.

The ODF of the present invention may further comprise at least one plasticizer. Plasticizers are an important component as they improve extensibility property of the ODF to provide flexibility to the ODF to minimize breakages that may occur under normal handling conditions as discussed in Example 1 below. Exemplary plasticizers may comprise polyethylene glycol (PEG), glycerol, Tween 20, phthalate derivatives (e.g., dimethyl phthalate, diethyl phthalate, dibutyl phthalate), citrate derivatives (e.g., tributylcitrate, triethylcitrate, acetyl citrate, citric acid), glycerol monoacetate, glycerol diacetate, triacetate, triacetin, polysorbate, cetyl alcohol, 1,3 butanediol, 1,4 butanediol, sorbitol, sodium diethylsulfosuccinate, and castor oil. In some embodiments, examples of PEG range from PEG 200 to PEG 35,000 and, more specifically, PEG 6000, PEG 4000, PEG 3350, PEG 2000, PEG 1000 and PEG 400.¹

Table 2 below illustrates several preferred formulations of the ODF of the present invention as discussed in connection with Example 1. As Table 2 illustrate, certain combinations of film-forming polymers work better with each other along with certain plasticizers. The following are some of these embodiments:

In some embodiments that comprise HPMC 6 cps as the primary polymer, the ODF of the present invention further comprises a plasticizer with high viscosity above about 500 cps but below about 2000 cps and low molecular weight of below about 1000 Daltons. An exemplary plasticizer with such high viscosity and low molecular weight comprises glycerol.

In other embodiments that comprise HPMC 15 cps as the primary polymer, the ODF of the present invention further comprises a plasticizer with low viscosity below 200 cps but above 3 cps and high molecular weight of above about 5000 Daltons but below about 5,000,000 Daltons. An exemplary plasticizer with such low viscosity and high molecular weight comprises PEG6000.

In some embodiments, the primary polymer is HPMC 6 cps and the secondary polymer is pullulan or HPMC 3 cps, and the plasticizer is glycerol, present at a ratio of about 3:1:1 by weight.

In some embodiments, the primary polymer is HPMC 6 cps and the secondary polymer is PVP k-90, and the plasticizer is PEG 4000, present at a ratio of about 7:1:2 by weight. In some preferred embodiments, the primary polymer is HPMC 15 cps and the secondary polymer is HPMC 3 cps or HPMC 6 cps, and the plasticizer is glycerol or PEG 6000, present at a ratio of about 2:2:1 to about 7:1:2 by weight. ¹Molecular weight measurement is conducted according to methodology described in Neira-Velazquez M G, Rodriguez-Hernandez M R, Hernandez-Hernandez E, Ruiz-Martinez A R Y. Polymer Molecular Weight Measurement. Handbook of Polymer Synthesis, Characterization, and Processing, 1st ed. New York: John Wiley & Sons; 2013. p 355

In some embodiments, the primary polymer is HPMC 15 cps and the secondary polymer is HPMC 3 cps, HPMC 6 cps or pullulan, and the plasticizer is PEG 6000, present at a ratio of about 3:1:1 by weight.

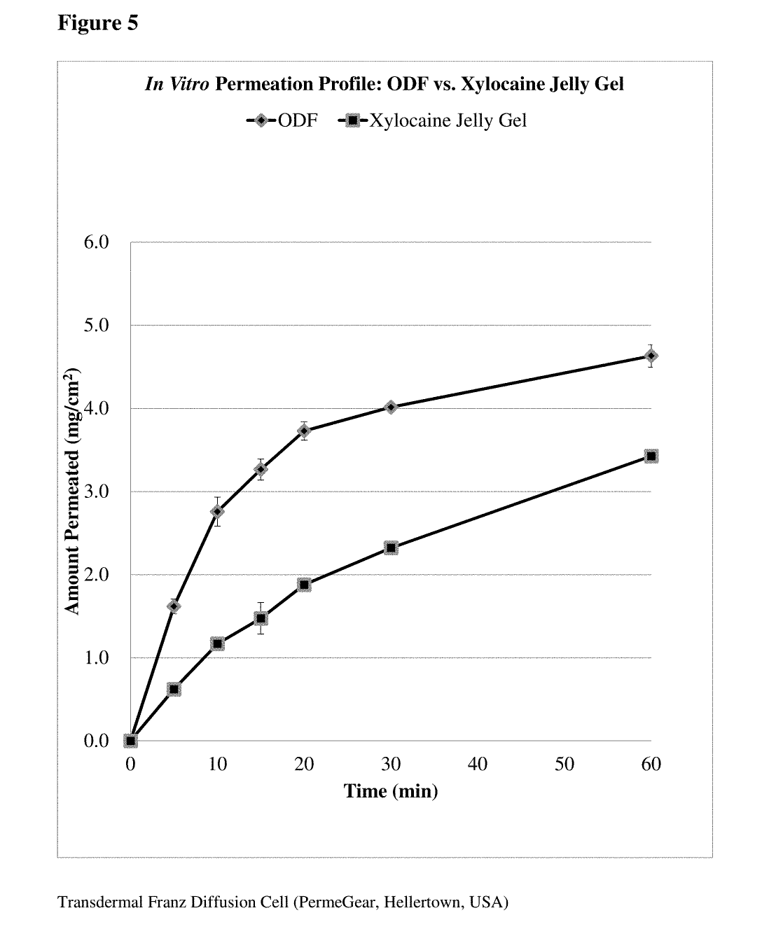

The fourth formulation listed in Table 2 was used to conduct dissolution and permeation studies comparing the ODF with currently commercially available orally administered lidocaine tablet, Trachisan Sore Throat Lozenges (Engelhard Arzenimittel GmbH & Co. KF, Germany) as well as gel, Xylocaine Jelly 2% (Recipharm Karlskoga AB, Sweden. The studies are discussed in further detail in Examples 2 and 3 below, respectively. Results of the dissolution studies are presented in FIGS. 1-4 comparing the ODF of the present invention to Trachisan Sore Throat Lozenges (Engelhard Arzenimittel GmbH & Co. KF, Germany) Results of the permeation studies are presented in FIG. 5 comparing the ODF of the present invention to Xylocaine Jelly 2% (Recipharm Karlskoga AB, Sweden).

As FIGS. 1-4 illustrate, the lidocaine ODF of the present invention is able to release about 90% of the lidocaine within between about 5 minutes to about 10 minutes after administration while the tablet requires about 30 minutes to release about 90% of the lidocaine in solutions of various pH in order to simulate various pH conditions of the human GI system. In addition, FIG. 5 illustrate that the ODF of the present invention provides permeation rate between about 0.8 to about 1.7 mg/cm² during the first 5 minutes of administration which is up to about 3 times higher than the gel. Therefore, both studies illustrate that the ODF of the present invention dissolves faster and permeates at a higher rate than currently available commercial orally administered lidocaine products such as Trachisan Sore Throat Lozenges and Xylocaine Jelly.

Furthermore, any of the foregoing embodiments may further comprise one or more fillers which may be any pharmaceutically acceptable filler. Examples of the fillers include, but not limited to, calcium phosphate, calcium sulfate, powdered sugar, silicates, dextrose, fructose, glucose, lactose, kaolin, starch, sucrose, maltose, mannitol, sorbitol, microcrystalline cellulose, powdered cellulose or any combination of the foregoing. In some embodiments, the filler is maltose, mannitol, or a combination thereof. In some embodiments, the filler consists of a mixture of water soluble and water insoluble fillers.

In any of the foregoing embodiments, a provided orally disintegrating film of local anesthetic may contain one or more active agents, e.g., pharmaceutical agent, nutraceutical agent, cosmetic agent, supplement. In some embodiments, the active agent is included in an amount from about 0.001% to about 60% based on the weight of all the components of the film. In other embodiments, the active agent is included in an amount from about 0.1% to 45% based on the weight of all the components of the film. In yet other embodiments, the active agent is included in an amount from about 1% to 40% based on the weight of all the components of the film.

Furthermore, other components can be added to provided films, including but not limited to, sweeteners, flavoring agents, coloring agents, saliva stimulants, taste masking agents, surfactants, preservatives, anti-foam agents, penetration enhancers, saliva stimulating agents, dispersants, buffering agents, thickening agents, enzyme inhibitors, and solubilizers.

In certain embodiments, a provided film comprises a sweetener. Sweeteners can be used to improve palatability and are usually classified as natural or artificial sweeteners. Exemplary natural sweeteners include, but are not limited to, dextrose, fructose, glucose, liquid glucose, maltose, rebiana, glycyrrhizin, thaumatin, sorbitol, mannitol, isomalt, maltitol, xylitol, and erythritol. Exemplary artificial sweeteners include, but are not limited to, saccharin, cyclamate, aspartame, acesulfame-K, sucralose, alitame and neotame. In certain embodiments, a sweetener represents about 0% to about 30% based on the dry weight of all the components of the film. In certain embodiments, a sweetener represents about 0.1% to about 25% based on the dry weight of all the components of the film. In certain embodiments, a sweetener represents about 1% to about 10% based on the dry weight of all the components of the film. In certain embodiments, a sweetener represents about 1% to about 7% based on the dry weight of all the components of the film. In certain embodiments, a sweetener represents about 1% to about 6% based on the dry weight of all the components of the film. In certain embodiments, a sweetener represents about 1% to about 5% based on the dry weight of all the components of the film.

In certain embodiments, a provided film comprises a flavoring agent. Examples of the flavoring agent may include, but are not limited to flavor oils such as peppermint oil, cinnamon oil, spearmint oil and oil of nutmeg, and flavor essence extracted from vanilla, cocoa, coffee and chocolate, and fruit essence obtained from apple, raspberry, cherry, pineapple and other citrus fruits such as orange, lemon and lime. Specific examples of the sweetening agent used in the present invention may include saccharine, sucrose, fructose, glucose, sucralose and mannitol. In certain embodiments, a provided film comprises a coloring agent. Coloring agents can be added to enhance the aesthetic appeal of the oral film, especially when formulation ingredients or drugs are presented in insoluble or suspension form. Exemplary coloring agents may include, but are not limited to Food Drug and Cosmetic (FD&C) colors such as FD&C Blue 1 Aluminum Lake, FD&C Yellow 5 Aluminum Lake, FD&C Yellow No. 6 Lake or any other pharmaceutically acceptable color additives that impart colors when added to the pharmaceutical composition. In certain embodiments, a colorant represents 0% to about 1% based on the dry weight of all the components of the film. In certain embodiments, a colorant represents about 0.001% to about 1% based on the dry weight of all the components of the film.

Other examples of the pharmaceutically acceptable excipients or additives commonly known to those skilled in the art may also be optionally added with the active ingredient as needed.

In certain embodiments, the ODF of the present invention may comprise a saliva stimulant. Saliva stimulants can be added to increase the rate of saliva production in order to promote a faster disintegration of the orodispersible film. Exemplary saliva stimulants include, but are not limited to, acidic compounds as citric acid, malic acid, lactic acid, ascorbic acid and tartaric acid. In other embodiments, some sweeteners can be used as saliva stimulants, including but not limited to glucose, fructose, xylose, maltose, and lactose. In certain embodiments, a saliva stimulant represents about 0% to about 10% based on the dry weight of all the components of the film. In certain embodiments, a saliva stimulant represents about 0% to about 7% based on the dry weight of all the components of the film. In certain embodiments, a saliva stimulant represents 0% to about 6% based on the dry weight of all the components of the film. In certain embodiments, a saliva stimulant represents about 2% to about 6% based on the dry weight of all the components of the film.

In certain embodiments, a provided film comprises taste-masking agents. Taste-masking agents can be added to ameliorate the organoleptic characteristics of the film. In certain embodiments, taste masking agents may be used to mask unpleasant taste of some components. Exemplary of taste-masking agents include, but are not limited to, cyclodextrins, maltodextrins, ion-exchange resins, amino acids, gelatin, gelatinized starch, liposomes, lecithins or lecithin-like substances and salts. In certain embodiments, the taste masking agent comprises about 0% to about 15% based on the dry weight of all the components of the film. In certain embodiments, the taste masking agent represents 0% to about 10% based on the dry weight of all the components of the film. In certain embodiments, the taste masking agent represents about 0% to about 7.5% based on the dry weight of all the components of the film. In certain embodiments, the taste masking agent represents about 0% to about 5% based on the dry weight of all the components of the film.

In certain embodiments, the ODF of the present invention may comprise a surfactant. Surfactants are surface-active agents that lower surface tension and thereby increase the emulsifying, foaming, dispersing, spreading and wetting properties of a product. Exemplary edible surfactants include, but are not limited to, sorbitan fatty acid esters (e.g., sorbitan monoisostearate, sorbitan monolaurate, sorbitan monooleate, sorbitan monopalmitate, sorbitan monostearate, sorbitan sesquistearate, sorbitan sesquioleate, sorbitan trilaurate, sorbitan trioleate, sorbitan tristearate), sucrose palmitate, glyceryl monooleate, vitamin E, polyethylene glycol succinate, propylene glycol monolaurate, myristyl alcohol, polyoxyethylene alkyl ethers, polyoxyethylene castor oil derivatives, sodium lauryl sulfate, and propylene glycol dilaurate. In certain embodiments, a surfactant represents about 0.01% to about 5% based on the dry weight of all the components of the film. In certain embodiments, a surfactant represents about 0.4% to about 0.7% based on the dry weight of all the components of the film.

In certain embodiments, the ODF of the present invention may comprise a dispersant for the film forming polymer. Film forming polymers are often supplied as a solution containing dispersants for maintaining stability of the film forming polymer dispersion. For example, polyvinyl acetate can be supplied with dispersants such as sodium lauryl sulfate and povidone. As another example, methacrylate copolymer can be supplied with macrogol cetostearyl ether and sodium lauryl sulfate, or sorbic acid and sodium hydroxide as dispersants. The dispersant(s) typically are present in amounts from 0.001% to 10% based on the dry weight of all the components of the film. In certain embodiments, the dispersant is present in amounts ranging from 0.01% to 8% based on the dry weight of all the components of the film. In certain embodiments, a dispersant is present in amounts ranging from 0.1% to 5% based on the dry weight of all the components of the film. In certain embodiments, a dispersant represents about 0.001% to about 1% based on the dry weight of all the components of the film. In certain embodiments, a dispersant represents about 0.04% to about 0.7% based on the dry weight of all the components of the film. In certain embodiments, a dispersant represents about 0.01% to about 7.5% based on the dry weight of all the components of the film.

In certain embodiments, the ODF of the present invention may comprise a buffering agent. Buffering agents can be added to manipulate the pH. The pH is involved in the dissolution and stabilization of the components in the formulation, but also with their absorption through the oral mucosa. Exemplary buffer agents include, but are not limited to citrate buffers, phosphate buffers, acetate buffers, carbonate buffers, ammonia buffers, borate buffers, lactate buffers, ethanolamine buffers, glycine buffers, methionine buffers, glutamate buffers and succinate buffers. In certain embodiments the pH buffer is an acid/acid salt system. Exemplary acid/acid salt systems include, but are not limited to, citric acid/citric acid salts (e.g. sodium citrate, potassium citrate), citric acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), citric acid/tartaric acid salts (e.g. sodium tartrate, potassium tartrate), citric acid/boric acid salts (e.g. sodium borate, potassium borate), citric acid/malic acid salts (e.g. sodium malate, potassium malate), citric acid/maleic acid salts (e.g. sodium maleate, potassium maleate), tartaric acid/citric acid salts (e.g. sodium citrate, potassium citrate), tartaric acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), tartaric acid/tartaric acid salts (e.g. sodium tartrate, potassium tartrate), tartaric acid/boric acid salts (e.g. sodium borate, potassium borate), tartaric acid/malic acid salts (e.g. sodium malate, potassium malate), tartaric acid/maleic acid salts (e.g. sodium maleate, potassium maleate), boric acid/citric acid salts (e.g. sodium citrate, potassium citrate), boric acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), boric acid/tartaric acid salts (e.g. sodium tartrate, potassium tartrate), boric acid/boric acid salts (e.g. sodium borate, potassium borate), boric acid/malic acid salts (e.g. sodium malate, potassium malate), boric acid/maleic acid salts (e.g. sodium maleate, potassium maleate), malic acid/citric acid salts (e.g. sodium citrate, potassium citrate), malic acid/phosphoric acid salts (e.g. sodium aluminum phosphate, sodium monobasic phosphate, sodium dibasic phosphate, sodium tribasic phosphate, potassium tribasic phosphate, potassium monobasic phosphate, potassium dibasic phosphate), malic acid/tartaric acid salts (e.g. sodium tartrate, potassium tartrate), malic acid/boric acid salts (e.g. sodium borate, potassium borate), malic acid/malic acid salts (e.g. sodium malate, potassium malate), malic acid/maleic acid salts (e.g. sodium maleate, potassium maleate), maleic acid/citric acid salts (e.g. sodium citrate, potassium citrate), maleic acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), maleic acid/tartaric acid salts (e.g. sodium tartrate, potassium tartrate), maleic acid/boric acid salts (e.g. sodium borate, potassium borate), maleic acid/malic acid salts (e.g. sodium malate, potassium malate), maleic acid/maleic acid salts (e.g. sodium maleate, potassium maleate). In certain embodiments, the buffer system represents about 0% to about 15% by weight of the film. In certain embodiments, a buffer system represents 0% to about 10% by weight of the film. In certain embodiments, a buffer system represents about 0% to about 7.5% by weight of the film.

### Preparation of Orally Disintegrating Film

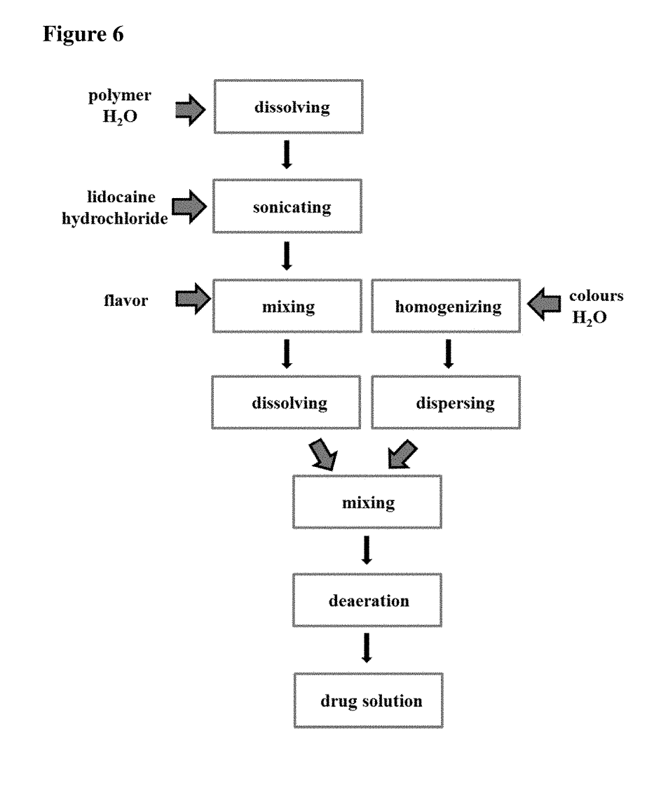

The present invention also provides a method for preparing the ODF of the present invention as illustrated by FIG. 6. The method preferably comprises the step of dissolving primary and secondary film forming polymers as well as plasticizer of the appropriate amount and ratio in water. Note that no organic solvents are required. In fact, manufacturing the ODF of the present invention does not require any organic solvents because the film-forming polymers of the present invention dissolve readily in water. Avoidance of organic solvents in the manufacture of an ODF is advantageous since even small amounts of organic solvents can be toxic to humans HPMC is particularly suitable as a film-forming polymer since it is a hydrophilic polymer that dissolves easier during manufacturing of the ODF, is more readily available and less inexpensive than other named film-forming polymers.

Next, lidocaine or a salt thereof such as lidocaine hydrochloride is also dissolved in the solution. In order to save production time, the solution may then undergo a sonication process the help eliminate air bubbles.

The solution is kept under rotation until the hydrophilic film forming polymers have completely dissolved and a homogeneous blend has been obtained. The solution is prepared in such a way as to form a pre-casting blend. Next, the solution is transferred to a surface of a suitable carrier material and dried to form the ODF. Examples of suitable carrier materials are non-siliconized polyethylene terephthalate film, non-siliconized paper, polyethylene-impregnated kraft paper or non-siliconized polyethylene film. Transfer of the solution onto the carrier material can be performed using any conventional film coating equipment. Drying of the film may preferably be carried out in a high-temperature air-bath using a drying oven, drying tunnel, vacuum, drier or any other suitable drying equipment known to those skilled in the art. For example, the film may be dried in an oven at about 80° C. over a period of about 20 minutes so as to form the orally administrable film of desired thickness and may then be cut into desired size.

In yet another embodiment, the method may further comprise the step of adding other ingredients such as one or more flavoring agent, sweetening agent and coloring agent to be dissolved or mixed with the pharmacologically active agent, hydrophilic film forming polymers and water soluble plasticizers in the method for preparing the film.

### Uses of Orally Disintegrating Film

The orally disintegrating film described herein may be of use in various situations including but not limited to local anesthetic for dental procedures such as oral surgery, tooth extraction, root canal as well as for alleviating pain caused by toothaches, oral ulcers, cold sores or teething. The ODF described herein can be administered orally to a site at or adjacent to a painful region or an area for oral surgery or dental treatment to ameliorate, eliminate or prevent pain. In the preferred embodiment, the ODF is designed to exert its local anesthetic effect by diffusion across the oral mucosa, thus offering an alternative route of local anesthetic administration as well as to relieve short-term local pain from sore throat. The ODF of the present invention can be administered once or multiple times per day.

In certain embodiments, a provided film may be administered to the oral mucosa or other mucous membranes where they are rapidly disintegrated by saliva and/or other aqueous materials on the mucosal surface. In certain embodiments, upon disintegration, a provided film releases one or more agents (e.g., pharmaceutical agent, nutraceutical agent, supplement, or cosmetic agent) to the mucous membranes. A provided film may be administered in such a manner so as to deliver a therapeutically effective amount of an agent.

In an embodiment, the lidocaine ODF of the present invention contains about 24 mg of lidocaine per ODF. It will be understood that the total daily usage of film described herein may be determined by an attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001).

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that, unless otherwise indicated, the entire contents of each of the references cited herein are incorporated herein by reference to help illustrate the state of the art. The following examples contain important additional information, exemplification and guidance which can be adapted to the practice of this invention in its various embodiments and the equivalents thereof.

These and other aspects of the present invention will be further appreciated upon consideration of the following Examples, which are intended to illustrate certain particular embodiments of the invention but are not intended to limit its scope, as defined by the claims.

### Example 1: Influence of Film Forming Polymers and Plasticizers on ODF Properties

Film forming polymers and plasticizers as well as their respective weight % in the ODF substantially affect properties of the ODF. Various formulations using film forming polymers such as hydroxypropyl methylcellulose (HPMC), hydroxyproply cellulose (HPC), povidone (PVP), pullulan and/or polyvinyl alcohol (PVA) in combination with plasticizers such as glycerol, Tween 20, PEG400, PEG4000 and/or PEG6000 were tested and evaluated in a study.

The properties of the ODF were evaluated based on four properties: A. film formation, B. film extensibility, C. film homogeneity and D. the smoothness of the film surface. To quantify film forming property results, a score of 1 indicates no indication of breakage in the film, and a score of 0 indicates the presence of breakage in the film. To quantify film extensibility results, a score of 1 indicates no indication of breakage upon pulling or bending, and a score of 0 indicates the presence of breakage upon pulling or bending. To quantify film homogeneity results, a score of 1 indicates no layer separation, and a score of 0 indicates the presence of layer separation. To quantify smoothness of surface results, a score of 1 indicates the absence of precipitation or air bubbles, and a score of 0 indicates the presence of precipitation or air bubbles. Table 1A lists examples of tested formulations of lidocaine ODF and their results.

The study was performed on ODF with only one film-forming polymer. As Table 1A illustrate below, ODF comprising only one film-forming polymer are not successful as not one formulation achieved a score of 4, indicating that other ingredients such as another film-forming polymer may be required to create a successful ODF. However, the results do indicate that HPC and HPMC tend to score higher.

TABLE 1A

| Polymer 1 | Polymer:Solvent | A | B | C | D |
|---|---|---|---|---|---|
| PVA | 1:5 | 0 | 0 | 0 | 0 |
| PVA | 1:4 | 0 | 0 | 0 | 0 |
| PVA | 3:10 | 0 | 0 | 0 | 0 |
| HPC | 1:5 | 0 | 0 | 0 | 1 |
| HPC | 1:4 | 1 | 0 | 1 | 1 |
| HPC | 3:10 | 1 | 0 | 1 | 1 |
| HPMC 3 cps | 1:5 | 0 | 0 | 0 | 0 |
| HPMC 3 cps | 1:4 | 0 | 0 | 0 | 0 |
| HPMC 3 cps | 3:10 | 1 | 0 | 0 | 0 |
| HPMC 6 cps | 1:5 | 1 | 0 | 1 | 1 |
| HPMC 6 cps | 1:4 | 1 | 0 | 1 | 1 |
| HPMC 6 cps | 3:10 | 1 | 0 | 1 | 1 |
| Pullulan | 1:5 | 0 | 0 | 0 | 0 |
| Pullulan | 1:4 | 0 | 0 | 0 | 0 |
| Pullulan | 3:10 | 0 | 0 | 0 | 0 |
| Povidone K-30 | 1:5 | 0 | 0 | 0 | 0 |
| Povidone K-30 | 1:4 | 0 | 0 | 0 | 0 |
| Povidone K-30 | 3:10 | 0 | 0 | 0 | 0 |
| Povidone K-90 | 1:5 | 0 | 0 | 0 | 0 |
| Povidone K-90 | 1:4 | 0 | 0 | 0 | 0 |
| Povidone K-90 | 3:10 | 0 | 0 | 0 | 0 |

| | |
|---|---|
| A: Film forming property | 1: No indication of breakage |
| | 0: Presence of breakage |
| B: Extensibility | 1: No indication of breakage upon pulling or bending |
| | 0: Presence of breakage upon pulling or bending |
| C: Homogeneity | 1: Absence of layer separation |
| | 0: Presence of layer separation |
| D: Smoothness of surface | 1: Absence of precipitation or air bubbles |
| | 0: Presence of precipitation or air bubbles |

Taking HPC and HPMC as primary polymers, additional studies were conducted with formulations containing the primary and as well as a secondary polymer. Some of the more successful results are summarized in table 1B below. As seen in table 1B, inclusion of primary and secondary polymers yielded some better results, but the resulting ODF still lacked the extensibility property (column B).

TABLE 1B

| Primary | Seconary | Polymer | | | | |
|---|---|---|---|---|---|---|
| Polymer | Polymer | Ratio | A | B | C | D |
| HPMC 6cps | HPC | 1:1 | 1 | 0 | 1 | 1 |
| HPMC 6cps | HPMC 3cps | 1:1 | 1 | 0 | 1 | 1 |
| HPMC 6cps | HPMC 3cps | 3:2 | 1 | 0 | 1 | 1 |
| HPMC 6cps | PVA | 7:3 | 1 | 0 | 1 | 1 |
| HPMC 6cps | HPC | 7:3 | 1 | 0 | 1 | 1 |
| HPMC 6cps | Povidone K-30 | 7:3 | 1 | 0 | 1 | 1 |
| HPMC 6cps | PVA | 4:1 | 1 | 0 | 1 | 1 |
| HPMC 6cps | HPC | 4:1 | 1 | 0 | 1 | 1 |
| HPMC 6cps | Povidone K-90 | 9:1 | 1 | 0 | 1 | 1 |
| HPC | HPMC 6cps | 3:2 | 1 | 0 | 1 | 1 |
| HPC | PVA | 7:3 | 1 | 0 | 1 | 1 |
| HPC | HPMC 6cps | 7:3 | 1 | 0 | 1 | 1 |
| HPC | Povidone K-30 | 7:3 | 1 | 0 | 1 | 1 |
| HPC | HPMC 3cps | 4:1 | 1 | 0 | 1 | 1 |
| HPC | Povidone K-30 | 4:1 | 1 | 0 | 1 | 1 |
| HPC | PVA | 9:1 | 1 | 0 | 1 | 1 |
| HPC | Povidone K-30 | 9:1 | 1 | 0 | 1 | 1 |
| HPC | Povidone K-90 | 9:1 | 1 | 0 | 1 | 1 |

A: Film forming property 1: No indication of breakage 0: Presence of breakage
B: Extensibility 1: No indication of breakage upon pulling or bending 0: Presence of breakage upon pulling or bending
C: Homogeneity 1: Absence of layer separation 0: Presence of layer separation
D: Smoothness of surface 1: Absence of precipitation or air bubbles 0: Presence of precipitation or air bubbles

To overcome the extensibility problem, plasticizer such as the higher molecular weight PEG (PEG 4000 or PEG 6000) or low molecular weight PEG (PEG 400), glycerol and Tween 20 was incorporated into the formulation. The incorporation of the plasticizer to the formulation in the presence of polymers substantially improved the resulting ODF. Table 2 lists several of the more successful formulations.

TABLE 2

| Primary | Secondary | | | | | | |
|---|---|---|---|---|---|---|---|
| Polymer | Polymer | Plasticizer | Ratio | A | B | C | D |
| HPMC 6cps | HPMC 3cps | Glycerol | 3:1:1 | 1 | 1 | 1 | 1 |
| HPMC 6cps | Pullulan | Glycerol | 3:1:1 | 1 | 1 | 1 | 1 |
| HPMC 6cps | PVP K-90 | PEG 4000 | 7:1:2 | 1 | 1 | 1 | 1 |
| HPMC 15cps | HPMC 3cps | Glycerol | 2:2:1 | 1 | 1 | 1 | 1 |
| HPMC 15cps | HPMC 3cps | PEG 6000 | 3:1:1 | 1 | 1 | 1 | 1 |
| HPMC 15cps | HPMC 6cps | PEG 6000 | 3:1:1 | 1 | 1 | 1 | 1 |
| HPMC 15cps | Pullulan | PEG 6000 | 3:1:1 | 1 | 1 | 1 | 1 |
| HPMC 15cps | HPMC 6cps | PEG 6000 | 7:1:2 | 1 | 1 | 1 | 1 |

A: Film forming property 1: No indication of breakage 0: Presence of breakage
B: Extensibility 1: No indication of breakage upon pulling or bending 0: Presence of breakage upon pulling or bending
C: Homogeneity 1: Absence of layer separation 0: Presence of layer separation
D: Smoothness of surface 1: Absence of precipitation or air bubbles 0: Presence of precipitation or air bubbles

Among all tested polymers, HPMC, povidone (PVP), and pullulan appear suitable for use in making ODF. Preferably, HPMC 6 cps and HPMC 15 cps may be used as the primary polymer. As noted in the Preparation of ODF section, HPMC polymers in particular provide the additional advantage of lower cost, is readily available and faster dissolution properties in water in comparison to other film-forming polymers named here. Therefore, in some embodiments, HPMC polymers may be used for both primary as well as secondary polymers. Suitable plasticizers include PEG 6000, PEG 4000, PEG 400, glycerol and Tween 20.

In the following two Examples, dissolution study and permeation study were conducted to compare the lidocaine ODF of formulation 4 in Table 1B above with lidocaine tablets, Trachisan Sore Throat Lozenges (Engelhard Arzenimittel GmbH & Co. KF, Germany) as well as gel, Xylocaine Jelly 2% (Recipharm Karlskoga AB, Sweden).

### Example 2: In-Vitro Dissolution Study of Lidocaine ODF

Dissolution study of the film was carried out in USP Apparatus IIV (Rotating Basket Apparatus) using 0.1N HCl (pH 1.2), phosphate buffers (pH 4.5 and 6.8) and water at 37.0±0.5° C. as dissolution media at 50 rotations per minute (rpm) to simulate in-vivo condition in stomach, duodenum, and jejunum.

Lidocaine ODF and the tablet, Trachisan Sore Throat Lozenges (Engelhard Arzenimittel GmbH & Co. KF, Germany)) were placed in the dissolution media. 5 ml aliquots of the samples were collected over a period of an hour at 3, 6, 9, 12, 15, 30, 45 and 60 minutes. The aliquots were assayed for drug content at 230 nm wavelength using UV-spectrophotometer. The cumulative percentage drug release was calculated to establish the dissolution profiles as shown in FIGS. 1-4.

All experiments were each carried out N=3 times. It should be noted that dissolution profiles can plateau slightly above or below 100% due to slight lidocaine content variations. In all cases, plateauing of the dissolution profile curves should indicate completion of dissolution. The results of dissolution study (FIGS. 1-4) revealed that the lidocaine ODF is able to release 90% of the lidocaine within about 5 to about 10 minutes while the tablet requires about 30 minutes to release 90% of the lidocaine.

### Example 3: Permeation Study of Lidocaine ODF

The in vitro permeation study of the film was conducted by using the Transdermal Franz Diffusion Cell, in which Lidocaine ODF and the gel, Xylocaine Jelly 2% (Recipharm Karlskoga AB, Sweden) diffusing from the donor compartment through the artificial skin to the recipient compartment was used to simulate drug diffusion across the oral mucosa. The receptor compartment was conditioned at 37.0+/−0.5 C with deionized water to simulate the physiological environment of the oral cavity. Three samples (N=3) of 0.99 to 1 ml were collected over a period of one hour at 5, 10, 15, 20, 30, and 60 minutes for HPLC analysis. FIG. 5 illustrates the permeation results that demonstrate that the lidocaine ODF has a substantially higher permeation rate than the gel. For example, FIG. 5 shows that the permeation of lidocaine ODF is about twice that of the gel at the 30 min mark.

Although the present invention has been described in terms of specific exemplary embodiments and examples, it can be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

It is to be understood that both the foregoing general description and the following detailed description are exemplary and explanatory only and are not restrictive of the invention, as claimed.

These and other changes can be made to the technology in light of the detailed description. In general, the terms used in the following disclosure should not be construed to limit the technology to the specific embodiments disclosed in the specification, unless the above detailed description explicitly defines such terms. Accordingly, the actual scope of the technology encompasses the disclosed embodiments and all equivalent ways of practicing or implementing the technology.

What is claimed is:

1. A fast acting orally disintegrating film (ODF) comprising:
lidocaine free-base or a pharmaceutically acceptable salt thereof;
at least one primary film-forming polymer;
at least one secondary film-forming polymer; and
at least one plasticizer,
wherein the primary film-forming polymer and the secondary film-forming polymer are present at a ratio of about 1:1 to about 20:1 by weight and
wherein the primary film-forming polymer and the secondary film-forming polymer are hydrophilic.

2. The composition of claim 1, wherein the lidocaine salt comprises lidocaine hydrochloride.

3. The composition of claim 1, wherein the lidocaine is present at an amount comprising at least about 10% to about 60% by dry weight of said film.

4. The composition of claim 1, wherein the primary film-forming polymer comprises hydroxylpropyl cellulose (HPC) or hydroxylpropyl methylcellulose (HPMC).

5. The composition of claim 1, wherein the secondary film-forming polymer comprises HPC, HPMC, pullulan and/or povidone (PVP).

6. The composition of claim 4, wherein the HPMC comprises HPMC 3 cps, HPMC 6 cps or HPMC 15 cps.

7. The composition of claim 5, wherein the PVP comprises PVP K-30 or PVP K-90.

8. The composition of claim 1, wherein the at least one plasticizer comprises polyethylene glycol (PEG), glycerol or Tween 20.

9. The composition of claim 8, wherein the PEG comprises PEG 400, PEG 4000 and/or PEG 6000.

10. The composition of claim 1, wherein the dry weight of the primary polymer as compared to dry weight of the secondary polymer is in a ratio of about 1:1 to about 7:1.

11. The composition of claim 1, wherein the dry weight of the primary and secondary polymers as compared to the dry weight of the plasticizer are in a ratio of about 4:1.

12. The composition of claim 1, wherein the primary polymer comprises HPMC 6 cps and the plasticizer comprises a plasticizer with high viscosity above about 500 cps but below about 2000 cps and low molecular weight of below about 1000 Daltons.

13. The composition of claim 1, wherein the primary polymer comprises HPMC 15 cps and the plasticizer comprises a plasticizer with low viscosity below about 200 cps but above about 3 cps and high molecular weight of above about 5000 Daltons but below about 5,000,000 Daltons.

14. The composition of claim 1, wherein the primary polymer comprises HPMC 15 cps, the secondary polymer comprises HPMC 3 cps or HPMC 6 cps and the plasticizer comprises glycerol or PEG 6000, wherein the primary film-forming polymer, the secondary film-forming polymer and the plasticizer are present at a ratio of about 2:2:1 to about 7:1:2 by weight.

15. The composition of claim 1, wherein the primary polymer comprises HPMC 15 cps, the secondary polymer comprises HPMC 3 cps, HPMC 6 cps or pullulan and the plasticizer comprises PEG 6000, wherein the primary film-forming polymer, the secondary film-forming polymer and the plasticizer present at a ratio of about 3:1:1 by weight.

16. The composition of claim 1, wherein the primary polymer comprises HPMC 6 cps, the secondary polymer comprises HPMC 3 cps or pullulan and the plasticizer comprises glycerol wherein the primary film-forming polymer, the secondary film-forming polymer and the plasticizer present at a ratio of about 3:1:1 by weight.

17. The composition of claim 1, wherein the primary polymer comprises HPMC 6 cps, the secondary polymer comprises PVP K-90 and the plasticizer comprises PEG 4000 wherein the primary film-forming polymer, the secondary film-forming polymer and the plasticizer present at a ratio of about 7:1:2 by weight.

18. The composition of claim 1, wherein the fast acting ODF disintegrates within about 60 seconds of administration.

19. The composition of claim 1, wherein the dissolution rate of the fast acting ODF allows for release of about 90% of the lidocaine within about 5 minutes to about 10 minutes of administration.

20. The composition of claim 1, wherein the permeation rate of the fast acting ODF allows about 0.8 to about 1.7 mg/cm² of lidocaine to permeate through the targeted area treated with the ODF within about 5 minutes of administration.

21. The composition of claim 1, wherein no trace of organic solvent is present in the composition.

22. The composition of claim 1, wherein the orally disintegrating film is produced by a method comprising the steps of:
mixing the local anesthetic, the two polymers and the at least one plasticizer in an aqueous solution;
transferring the aqueous solution to a surface of a suitable carrier material; and
drying the aqueous solution on surface of the carrier material to form a film.

23. The composition of claim 19, wherein the orally disintegrating file is produced without any organic solvent.

24. A method for providing local anesthesia to a patient in need, the method comprising: orally administering to the patient in need thereof an effective amount of a composition according to claim 1.

25. The method of claim 18, wherein said method is used during oral surgery or dental treatment.
